(19) United States
(12) Patent Application Publication
(10) Pub. No.: US 2024/0398387 A1
(43) Pub. Date: Dec. 5, 2024

(54) Systems and Methods for Reverberation Clutter Artifacts Suppression in Ultrasound Imaging

(71) Applicant: Mayo Foundation for Medical Education and Research, Rochester, MN (US)

(72) Inventors: Shigao Chen, Rochester, MN (US); Ping Gong, Rochester, MN (US); U Wai Francisco Lok, Rochester, MN (US); Joshua D. Trzasko, Rochester, MN (US)

(21) Appl. No.: 18/573,877

(22) PCT Filed: Jun. 20, 2022
(86) PCT No.: PCT/US2022/034182

§ 371 Date: Dec. 22, 2023

Related U.S. Application Data
(60) Provisional application No. 63/214,002, filed on Jun. 23, 2021.
(60) Provisional application No. 62/214,002, filed on Sep. 3, 2015.

(51) Int. Cl. A61B 8/08; A61B 8/00; G06T 5/50; G06T 7/00

(52) U.S. Cl. CPC A61B 8/5276; A61B 8/469; G06T 5/50; G06T 7/0012; G06T 2207/10136; G06T 2207/20004; G06T 2207/20224; G06T 2207/30004

(57) ABSTRACT

Systems and methods are provided to adaptively suppress reverberation clutter signals in ultrasound imaging. A robust principal component analysis (RPCA) may be used to separate a static or low dimension background signal from sparse, moving or high dimension objects in the presence of outliers. A tissue signal may be transformed to the wavelet domain to fulfill the sparsity conditions of RPCA. The use of the RPCA combined with wavelet kernels may be used to suppress reverberation clutter signals to achieve robust ultrasound attenuation coefficient estimation.

### CROSS-REFERENCE TO RELATED APPLICATIONS

This application claims the benefit of U.S. Provisional Patent Application Ser. No. 63/214,002 filed on Jun. 23, 2021 and entitled “Systems and Methods for Reverberation Clutter Artifacts Suppression in Ultrasound Imaging,” which is incorporated herein by reference as if set forth in its entirety for all purposes.

### STATEMENT REGARDING FEDERALLY SPONSORED RESEARCH

N/A

### BACKGROUND

Increasing fat content for some subjects may lead to liver steatosis, which may progress to fibrosis, cirrhosis, liver failure, or even hepatocellular carcinoma. One of the methods to evaluate fat content in the liver is proton density fat fraction (PDFF) acquired with magnetic resonance imaging (MRI) which was often used as a benchmarking standard. However, one of the limitations of MRI is low accessibility, thus prohibiting frequent follow-up exams. Ultrasound attenuation coefficient has also been shown to have the potential to quantify fat content in the human liver. Ultrasound attenuation coefficient estimation (ACE) has been reported using different approaches, such as spectral shift technique, reference phantom-based methods, and the reference frequency method (RFM). However, in ultrasound imaging the ultrasound pulses undergo multi-path reflections (for example, within the fat and muscle layers of body wall) that will reveal reverberation clutter artifacts at different locations. The reverberation clutters superimposed on liver echoes may bias the value of the attenuation coefficient, thus effective reverberation clutter suppression is needed for ACE.

Several methods have been proposed to suppress the reverberation clutter signals by transforming the received ultrasound signals to predefined bases that are orthonormal and independent such as Discrete Fourier Transform and Discrete Wavelet Transform, and then the pre-defined bases are filtered/discarded to mitigate the reverberation clutter signals. Although these approaches may be computation-effective, they may produce poor results when reverberation clutter and tissue characteristics are overlapped. Furthermore, physiological differences among patients imply large variability of signal characteristics, results in difficulty to choose appropriate thresholds/bases to suppress reverberation clutter signals. Also, conventional methods for selecting bases depends on the actual data.

Therefore, there remains a need for effective, adaptive methods to overcome these limitations in reverberation clutter suppression.

### SUMMARY OF THE DISCLOSURE

The present disclosure addresses the aforementioned drawbacks by providing systems and methods to suppress reverberation clutter signals adaptively. In some configurations, a robust principal component analysis (RPCA) may be used to separate a static (e.g. low dimension) background from sparse moving (e.g. high dimension) objects with the presence of outliers. Principal component analysis (PCA) may include computing a set of linearly uncorrelated variables which is called principal components based on the covariance characteristics of the data. While PCA may be used to suppress reverberation clutter signals adaptively, PCA may be sensitive to data outliers, and thus degrades the reverberation clutter suppression capability. RPCA, by contrast, may effectively suppress reverberation clutter signals in the presence of outliers.

In some configurations, RPCA may assume the sparsity nature of the high dimensional moving objects signals, and a transformation may be used to ensure the tissue signal satisfies the sparsity property. To handle the sparsity requirement of RPCA, the tissue signal may be transformed to the wavelet domain to fulfill the sparsity condition. In accordance with the present disclosure, the use of the RPCA combined with wavelet kernels, may be used to suppress reverberation clutter signals to achieve robust ACE.

In one configuration, a method is provided for reverberation signal suppression in ultrasound imaging of a subject. The method includes accessing or acquiring ultrasound imaging data of a subject that includes a plurality of frames at different times and including tissue signals and reverberation signals. The method also includes generating a region of interest (ROI) frames subset by determining a ROI for each frame in the plurality of frames and generating a spatiotemporal matrix from the ROI frames subset. The method also includes separating tissue signals from reverberation signals in the spatiotemporal matrix using an adaptive method. The method also includes generating an image of the subject with the reverberation signals suppressed by subtracting the separated reverberation signals from the ultrasound imaging data.

In one configuration, a system is provided for reverberation signal suppression in ultrasound imaging of a subject. The system includes a computer system configured to: i) access ultrasound imaging data of a subject that includes a plurality of frames at different times and including tissue signals and reverberation signals; ii) generate a region of interest (ROI) frames subset by determining a ROI for each frame in the plurality of frames; iii) generate a spatiotemporal matrix from the ROI frames subset; iv) separate tissue signals from reverberation signals in the spatiotemporal matrix using an adaptive method; and v) generate an image of the subject with the reverberation signals suppressed by subtracting the separated reverberation signals from the ultrasound imaging data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a block diagram of a non-limiting example ultrasound data acquisition.

FIG. 2 is a flowchart of non-limiting example steps is shown for a method 200 to adaptively suppress reverberation clutter signals in ultrasound imaging

FIG. 3A is an image depicting a non-limiting example region of interest of a B mode image of a tissue-mimicking phantom without added reverberation clutter signals.

FIG. 3B depicts non-limiting example reverberation clutter signals.

FIG. 3C depicts the non-limiting example of FIG. 3A mixed with the reverberation clutter signals of FIG. 3B. p FIG. 3D depicts the non-limiting example of FIG. 3A with the reverberation clutter signals suppressed in accordance with the present disclosure.

FIG. 4A is a non-limiting example B mode image of a tissue-mimicking phantom computed without any reverberation clutter signals.

FIG. 4B is a 2-D attenuation coefficient map fusion with the B mode image of FIG. 4A without reverberation signal suppression.

FIG. 4C is a 2-D attenuation coefficient map fusion with the B mode image of FIG. 4A with reverberation signal suppression.

FIG. 5A is a graph of a non-limiting example correlation between PDFF and estimated attenuation coefficient obtained from fifteen patients, without applying reverberation signal suppression.

FIG. 5B is a graph of the non-limiting example of FIG. SA showing the correlation between PDFF and estimated attenuation coefficient with applying reverberation signal suppression.

FIG. 6 is a block diagram of a non-limiting example ultrasound system that can implement the methods described in accordance with the present disclosure.

### DETAILED DESCRIPTION

Systems and methods are provided to adaptively suppress reverberation clutter signals in ultrasound imaging. In some configurations, a robust principal component analysis (RPCA) may be used to separate a static or low-dimension background signal from sparse, moving or high-dimension objects in the presence of outliers. To handle sparsity requirements of RPCA, a tissue signal may be transformed to the wavelet domain, or another suitable sparse domain, to fulfill the sparsity condition. The use of the RPCA combined with wavelet kernels may be used to suppress reverberation clutter signals to achieve robust ultrasound attenuation coefficient estimation.

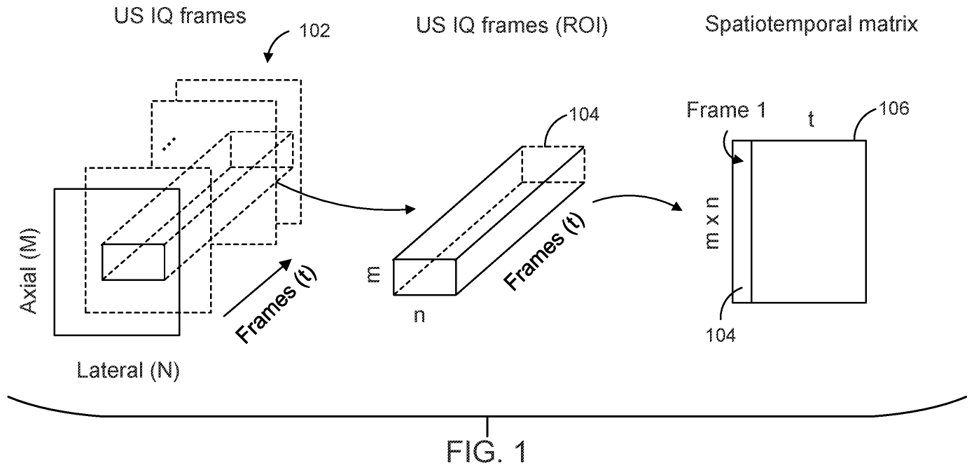

Referring to FIG. 1, a block diagram of a non-limiting example ultrasound data acquisition is shown. The ultrasound data may include in-phase/quadrature phase (IQ) data, or another format of ultrasound data such as post-beamformed radio frequency (RF) data, envelop data or pre-beamformed channel data, and the like. In the non-limiting example, a total of t consecutive frames 102 of ultrasound in-phase/quadrature-phase (IQ) data are acquired, with each frame 102 including of M×N ultrasound in-phase quadrature data (M row and N column). The region-of-interest 104 may be taken by selecting m×n of ultrasound IQ data for each frame 102. The ultrasound IQ spatiotemporal data are reshaped into a two-dimensional (2D) spatiotemporal matrix 106 with a dimension of (mn×t), with each column representing one ultrasound frame. There is no restriction for the transmission scheme, one can use plane/diverging waves, focused beams focused at different depths or steered at different directions, and synthetic aperture imaging, and the like. The methods in accordance with the present disclosure can be applied to ultrasound data acquired in either fundamental or harmonic imaging mode.

To differentiate the tissue signals from the reverberation clutter signals, the tissue signals may have large motion variability as compared with the reverberation signals across frames. In a non-limiting example of liver imaging, to obtain moving tissue signals of liver multiple ultrasound frames may be acquired with subjects breathing freely or breathing heavily so that the liver moves significantly during real-time in-vivo scanning. To achieve nearly static reverberation clutter signals which may originate from the body wall, the ultrasound probe may be held tightly upon the subject's body surface so that the clutter signals do not change significantly during breathing. In another non-limiting example, the beating heart may present a moving tissue signal to facilitate separation from static clutter signals from the body wall. Since the moving tissue signals possess high dimensional signals and the static reverberation clutter signals possess low dimensional signals, the reverberation clutter signals can be separated from the tissue signals using adaptive methods.

In some configurations, RPCA may be used to estimate the reverberation clutter signals from the received signals. Once the reverberation clutter signals are estimated, the tissue signals can be estimated by subtracting the received signals from the estimated reverberation clutter signals. Any appropriate separation method may be used. In non-limiting examples, a model-based method (e.g. principal component analysis, singular value decomposition), non-parametric-based method, or blind source method (e.g. independent component analysis), and the like may be used for the separation method.

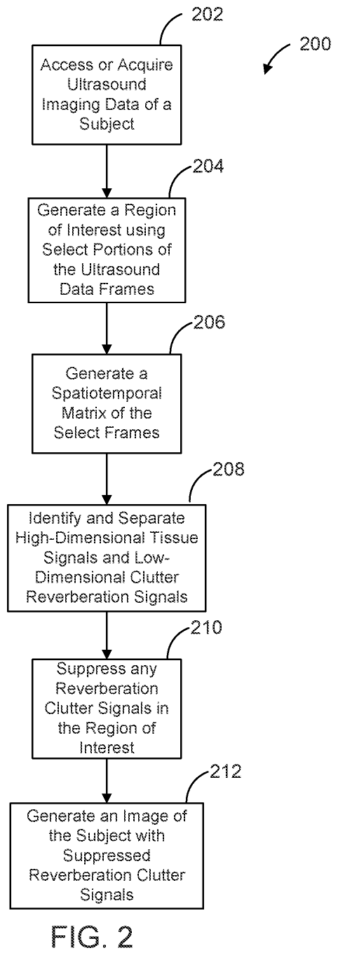

Referring to FIG. 2, a flowchart of non-limiting example steps is shown for a method 200 to adaptively suppress reverberation clutter signals in ultrasound imaging. Ultrasound imaging data of a subject may be accessed or acquired at step 202. In general, imaging data are spatiotemporal data. For instance, the imaging data may represent a time series of two-dimensional image frames or three-dimensional image volumes. For illustration purposes, the following teaching uses 2D image frames as a non-limiting example. The methods in accordance with the present disclosure can be applied to 2D image frames, 3D volume data, and the like. A region of interest (ROI) may be generated from a select set of frames of the ultrasound imaging data at step 204. A spatiotemporal matrix of the selected frames may be generated at step 206, and may be generated from entire frames, or from a portion of the frames that include the ROI. A spatiotemporal matrix may be generated as described in FIG. 1 where the ROI may be taken by selecting m×n of the ultrasound IQ data for each frame (M×N) where the frames are compiled over time (t). The ultrasound IQ spatiotemporal data are reshaped into a two-dimensional (2D) spatiotemporal matrix with a dimension of (mn×t), with each column representing one ultrasound frame. A spatiotemporal matrix may be used to display the similarity of the ultrasound frames in an ensemble and may be computed using pixels from the ROI, such as a lesion area.

In a non-limiting example, the ROI data-points may be transformed from 3-dimensional Cartesian coordinates to 2-dimensional Casorati co-ordinates, where each row and column represents the spatial and temporal data-points, respectively. The matrix can be quantitatively summarized by statistics (e.g., mean, median) to measure performance. Such performance metrics can be provided on a range of 0-1, 0%-100%, or another suitable range.

The high-dimensional tissue signals and low-dimensional reverberation signals may be identified and separated by processing the spatiotemporal matrix at step 208 using methods in accordance with the present disclosure. Reverberation clutter signals may then be suppressed in the region of interest at step 210. An image of the subject with suppressed reverberation clutter signals may also be generated at step 212.

A signal model for the observed received spatiotemporal IQ data can be expressed as:

$$X = S + L + N \qquad ( 1 )$$

Where X, S, L and N are received signals, tissue signals, reverberation clutter artifact, and noise. In (1), the reverberation (L) possesses static low-dimensional (low-rank) signals, while the tissue(S) possesses high-dimensional (high-rank) signals.

RPCA may be used to recover low-rank components and to reduce the impact of corrupted data. An RPCA technique may be expressed as follows:

$${min}_{\{ L , S \}} \{ {λ}_{1} {‖ L ‖}_{*} + {λ}_{2} {‖ S ‖}_{1} + \frac{1}{2} {‖ ( X - L - S ) ‖}_{F}^{2} \} \qquad ( 2 )$$

Where ∥L∥_{*}, ∥S∥₁, and ∥P∥_{F}² represent the nuclear norm, L1-norm and Frobenius norm of L, S and P. λ₁ and λ₂ are the regularization parameters which affect the estimated L and S. The method may be used in separating sparse dynamic data from static data, and may exploit the sparse property of signal S. In some configurations, the tissue signals S may not be sparse in the spatiotemporal domain, thus, making it difficult to meet the sparsity conditions. In a non-limiting example, the sparsity of the tissue signal in the wavelet-domain may be used to address sparsity condition issues instead of the spatiotemporal domain. Such an optimization problem can be expressed as

$${min}_{\{ L , S \}} \{ {λ}_{1} {‖ L ‖}_{*} + {λ}_{2} {‖ S ‖}_{1} + \frac{1}{2} {‖ ( X - L - {W}^{H} S ) ‖}_{F}^{2} \} \qquad ( 3 )$$

Where W^{H} is the adjoint 2D wavelet transformation. Any appropriate wavelet kernel may be used, and any wavelet kernel can be used for the 2D wavelet transformation and adjoint 2D wavelet transformation.

In some configurations, an Alternating Direction Method of Multiplier (ADMM) may be used as a possible optimization method to solve eq. (3). Any appropriate optimization algorithm may be used, such as such as augmented Lagrange multiplier, fast alternating minimization, iteratively reweighted least squares, and the like can be used to solve eq. (3). When using ADMM, two auxiliary variables U, and V may be introduced and eq. (3) may be rewritten as

$${min}_{\{ L , S \}} \{ {λ}_{1} {‖ U ‖}_{*} + {λ}_{2} {‖ V ‖}_{1} + \frac{1}{2} {‖ ( X - L - {W}^{H} S ) ‖}_{F}^{2} \} s . t . U = L , V = S \qquad ( 4 )$$

Removing the linear equality constraints in (4) with the Lagrangian method generates the following objective cost function:

$$C ( L , S , U , V , {E}_{1} , {E}_{2} ) = {λ}_{1} {‖ U ‖}_{*} + {λ}_{2} {‖ V ‖}_{1} + \frac{1}{2} {‖ ( X - L - {W}^{H} S ) ‖}_{F}^{2} + {μ}_{1} {‖ ( U - L - {E}_{1} ) ‖}_{F}^{2} + {μ}_{2} {‖ ( V - S - {E}_{2} ) ‖}_{F}^{2} \qquad ( 5 )$$

Where μ₁ and μ₂ are the ADMM optimization parameters to control the convergence of the algorithm. At each iteration k, ADMM may perform the following four steps to minimize the cost function

$${U}^{k + 1} = arg {min}_{{U}^{k}} [ {λ}_{1} {‖ {U}^{k} ‖}_{*} + {μ}_{1} {‖ ( {U}^{k} - {L}^{k} - {E}_{1} ) ‖}_{F}^{2} ] , \qquad ( 6 )$$

$${V}^{k + 1} = arg {min}_{{V}^{k}} [ {λ}_{2} {‖ {V}^{k} ‖}_{1} + {μ}_{2} {‖ ( {V}^{k} - {S}^{k} - {E}_{2} ) ‖}_{F}^{2} ] , \qquad ( 7 )$$

$${L}^{k + 1} = arg {min}_{{L}^{k}} [ \frac{1}{2} {‖ ( X - {L}^{K} - {W}^{H} {S}^{K} ) ‖}_{F} + {μ}_{1} {‖ ( {U}^{k} - {L}^{k} - {E}_{1} ) ‖}_{F}^{2} ] , \qquad ( 8 )$$

$${S}^{k + 1} = arg {min}_{{S}^{k}} [ \frac{1}{2} {‖ ( X - {L}^{K} - {W}^{H} {S}^{K} ) ‖}_{F} + {μ}_{2} {‖ ( {V}^{k} - {S}^{k} - {E}_{2} ) ‖}_{F}^{2} ] . \qquad ( 9 )$$

Eqs. (6) and (7) are convex problems possessing closed-form solutions: singular value thresholding (SVT) and soft thresholding (ST), respectively. Additionally, eqs. (8) and (9) can be solved by differentiating with respect to L^{k} and S^{k}, summarized as follows:

$${U}^{k + 1} = S V {T}_{\frac{{λ}_{1}}{{μ}_{1}}} ( {L}^{k} + {E}_{1}^{k} ) \qquad ( 10 )$$

$${V}^{k + 1} = S {T}_{\frac{{λ}_{2}}{{μ}_{2}}} ( {S}^{k} + {E}_{2} ) \qquad ( 11 )$$

$${L}^{k + 1} = \frac{1}{1 + {μ}_{1}} [ ( X - W {S}^{k} ) + {μ}_{1} ( {U}^{k} - {E}_{1}^{k} ) ] , \qquad ( 12 )$$

$${S}^{k + 1} = \frac{1}{1 + {μ}_{2}} [ ( ( X - {L}^{k} ) ) + {μ}_{2} ( {V}^{k} - {E}_{2}^{k} ) ] . \qquad ( 13 )$$

Where E₁ and E₂ can be computed using a gradient decent method. A non-limiting example method is summarized as the Table I below:

TABLE I
Non-limiting example method using ADMM

```
  Input Parameters. Input parameters are received spatiotemporal matrix,
X, and set the maximum iteration kₘₐₓ
  Initialize: L⁰ = 0, S⁰ = 0, U⁰ = 0, V⁰ = 0, λ₁, λ₂, μ₁, μ₂, E₁⁰, E₂⁰, k=0
  Main Iteration: Increment k by 1 and perform the following steps:
  Update U^{k+1} using eq. (10)
  Update V^{k+1} using eq. (11)
  Update L^{k+1} using eq. (12)
  Update S^{k+1} using eq. (13)
  Update E₁ and E₂ using gradient decent method
  Stop criteria: k > kₘₐₓ or ∥(X − L^{k} − W^{H}S^{k})∥_{F}² ≤ ε
  Output: The approximated solution of tissue signal is X−L^{k} obtained
after k iterations.
```

In some configurations, the RPCA algorithm may involve repeated computations of the singular value decomposition (SVD) and thresholding of matrices during the singular value thresholding process. This repeated computation of the SVD may be a bottleneck of computational complexity, but as one non-limiting example singular value thresholding may be used to alleviate this complexity by computing fewer singular values as those singular values that lie above a specified threshold. Speeding up the algorithms that involve thresholding of singular values may be accomplished by using a truncated SVD approach to compute only those singular values of interest. The truncated SVD approach is one non-limiting example of the possible methods for the fast computation of SVD, and any appropriate computation of SVD processes may be used.

### Non-Limiting Example B Mode Imaging

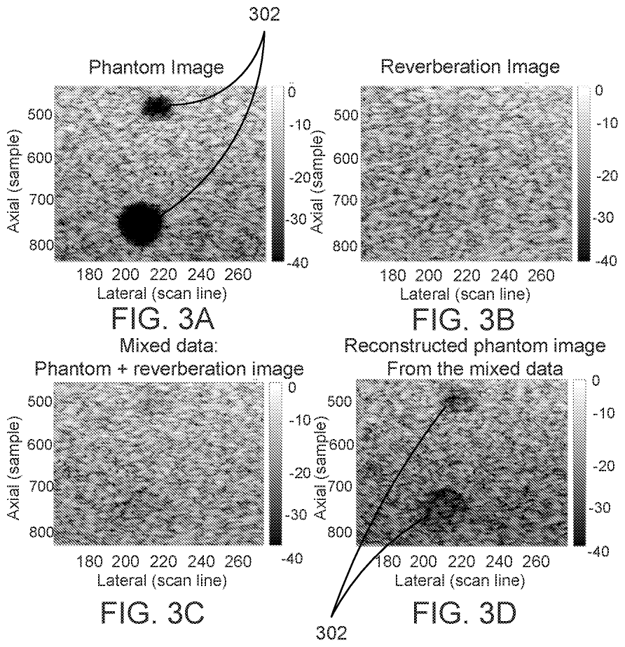

Referring to FIGS. 3A-D, a non-limiting example selected ROI of a 1^{st} frame of B mode image of a calibrated tissue-mimicking phantom is shown. Two hypoechoic cysts 302 were clearly visualized in the B mode image of FIG. 3A, which depicts the ROI of the 1^{st} frame of B image of the tissue-mimicking phantom without added reverberation clutter signals. FIG. 3B shows the selected ROI of the 1^{st} frame of B mode image of the reverberation clutter signals. FIG. 3C shows the selected ROI of the 1^{st} frame of B mode image of the calibrated tissue-mimicking phantom where the tissue signals were mixed with the reverberation clutter signals, and two hypoechoic cysts' signals were corrupted by the clutter signals. After applying reverberation clutter signal suppression, the two hypoechoic cysts can be better visualized than those of the mixed signals, as shown in FIG. 3D.

### Non-limiting Example Attenuation Coefficient Estimation

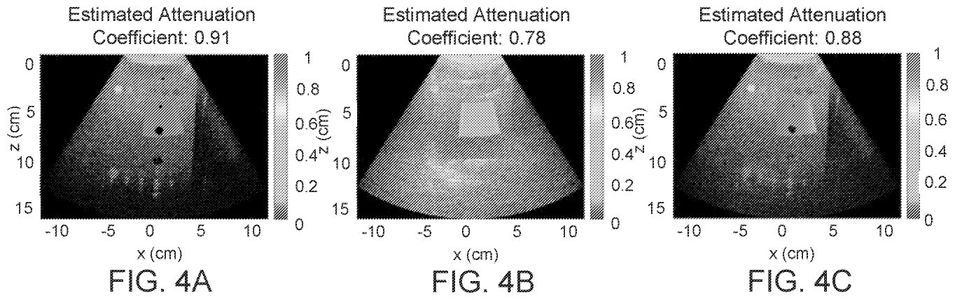

Referring to FIGS. 4A-4D, a non-limiting example of a 2-D attenuation coefficient map fusion is shown with FIG. 4A depicting the B-mode image of a tissue-mimicking phantom (with calibrated attenuation coefficient of 0.95 dB/cm/MHz) computed without any reverberation clutter signals. FIG. 4B and 4C show the 2-D attenuation coefficient map fusion with the B-mode image of a tissue-mimicking phantom without and with the reverberation signal suppression. The estimated attenuation coefficient values without and with reverberation signal suppression were 0.78 and 0.88, respectively. These results show that better attenuation coefficients can be estimated with reverberation signal suppression (estimated attenuation coefficient: 0.88 versus calibrated attenuation coefficient: 0.95) than that without reverberation signal suppression (estimated attenuation coefficient: 0.78 versus calibrated attenuation coefficient: 0.95).

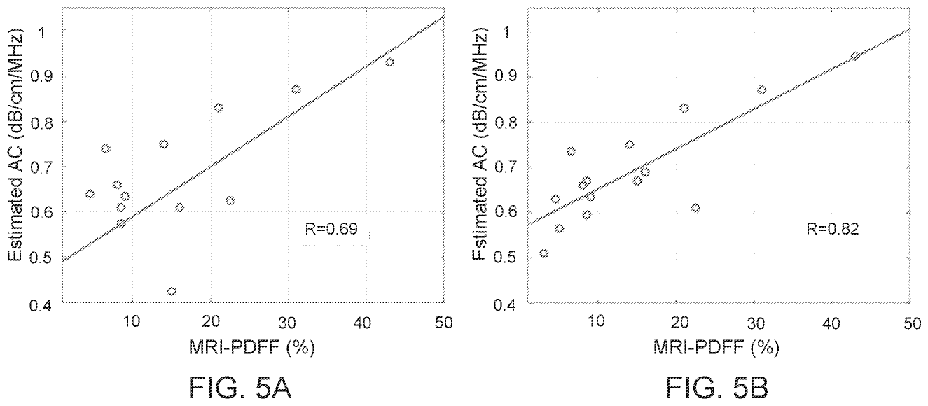

Referring to FIGS. 5A-B, non-limiting examples of correlations between proton density fat fraction (PDFF) acquired with MRI and ACE obtained in a fundamental mode during free breathing are shown. Patients breathed freely during data acquisition so that the liver moved significantly to facilitate separation of liver signals from unwanted static reverberation clutter signals from the body wall using the methods in accordance with the present disclosure. FIG. 5A shows the correlation between PDFF and estimated attenuation coefficient obtained from fifteen patients, without applying reverberation signal suppression. FIG. 5B shows the correlation between PDFF and estimated attenuation coefficient with applying reverberation signal suppression on the same fifteen patients. The Pearson's correlation coefficients in FIG. 5A and FIG. 5B were 0.69 and 0.82, respectively.

RPCA may be used to mitigate the reverberation clutter artifacts in ultrasound attenuation coefficient estimation. The methods in accordance with the present disclosure provide accurate and robust ultrasound attenuation coefficient estimation. In the non-limiting examples above, tissue moves while clutters are static. The methods in accordance with the present disclosure may also be used in situations where the unwanted clutter signals move while the desired tissue signal are static: in such cases, tissue signal will be low dimensional (low rank) and clutter signal will be high dimensional (high rank). Therefore, clutters can still be isolated and subtracted from the received signal to obtain a cleaner tissue signal to achieve clutter suppression.

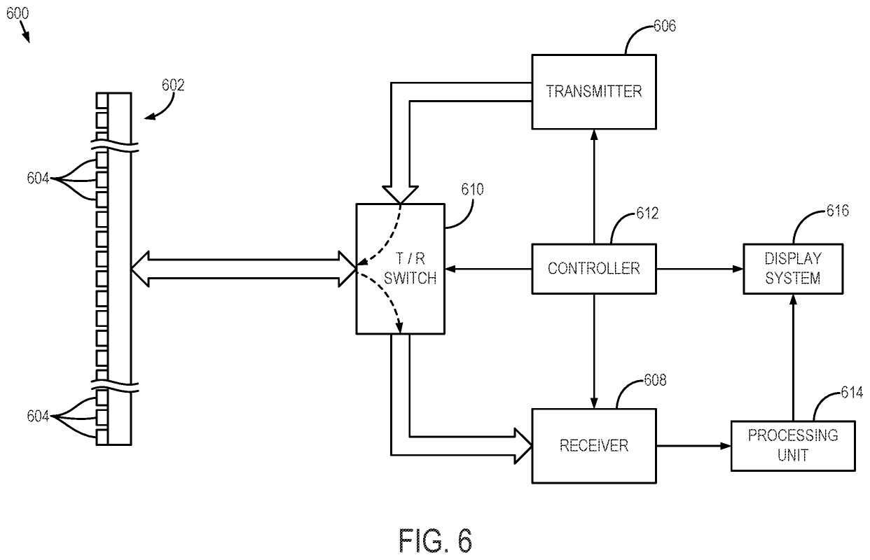

FIG. 6 illustrates an example of an ultrasound system 600 that can implement the methods described in the present disclosure. The ultrasound system 600 includes a transducer array 602 that includes a plurality of separately driven transducer elements 604. The transducer array 602 can include any suitable ultrasound transducer array, including linear arrays, curved arrays, phased arrays, and so on. Similarly, the transducer array 602 can include a 1D transducer, a 1.5D transducer, a 1.75D transducer, a 2D transducer, a 3D transducer, and so on.

When energized by a transmitter 606, a given transducer element 604 produces a burst of ultrasonic energy. The ultrasonic energy reflected back to the transducer array 602 (e.g., an echo) from the object or subject under study is converted to an electrical signal (e.g., an echo signal) by each transducer element 604 and can be applied separately to a receiver 608 through a set of switches 610. The transmitter 606, receiver 608, and switches 610 are operated under the control of a controller 612, which may include one or more processors. As one example, the controller 612 can include a computer system.

The transmitter 606 can be programmed to transmit unfocused or focused ultrasound waves. In some configurations, the transmitter 606 can also be programmed to transmit diverged waves, spherical waves, cylindrical waves, plane waves, or combinations thereof. Furthermore, the transmitter 606 can be programmed to transmit spatially or temporally encoded pulses.

The receiver 608 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 606 and the receiver 608 can be programmed to implement a high frame rate. For instance, a frame rate associated with an acquisition pulse repetition frequency (“PRF”) of at least 100 Hz can be implemented. In some configurations, the ultrasound system 600 can sample and store at least one hundred ensembles of echo signals in the temporal direction.

The controller 612 can be programmed to implement an imaging sequence using the techniques described in the present disclosure, or as otherwise known in the art. In some embodiments, the controller 612 receives user inputs defining various factors used in the implementation of the imaging sequence.

A scan can be performed by setting the switches 610 to their transmit position, thereby directing the transmitter 606 to be turned on momentarily to energize transducer elements 604 during a single transmission event. The switches 610 can then be set to their receive position and the subsequent echo signals produced by the transducer elements 604 in response to one or more detected echoes are measured and applied to the receiver 608. The separate echo signals from the transducer elements 604 can be combined in the receiver 608 to produce a single echo signal.

The echo signals are communicated to a processing unit 614, which may be implemented by a hardware processor and memory, to process echo signals or images generated from echo signals. As an example, the processing unit 614 can suppress reverberation signal clutter noise/artifacts using the methods described in the present disclosure. Images produced from the echo signals by the processing unit 614 can be displayed on a display system 616.

The present disclosure has described one or more preferred embodiments, and it should be appreciated that many equivalents, alternatives, variations, and modifications, aside from those expressly stated, are possible and within the scope of the invention.

1. A method for reverberation signal suppression in ultrasound imaging of a subject, comprising:
accessing ultrasound imaging data of a subject that includes a plurality of frames at different times and including tissue signals of a first dimension rank and reverberation signals of a second dimension rank;
generating a region of interest (ROI) frames subset by determining a ROI for each frame in the plurality of frames;
generating a spatiotemporal matrix from the ROI frames subset;
separating the tissue signals from the reverberation signals in the spatiotemporal matrix using an adaptive method; and
generating an image of the subject with the reverberation signals suppressed by subtracting the separated reverberation signals from the ultrasound imaging data.

2. The method of claim 1, wherein the adaptive method includes at least one of a robust principal component analysis (RPCA), principal component analysis, singular value decomposition, non-parametric-based method, independent component analysis, or blind source method.

3. The method of claim 2, wherein the adaptive method is RPCA, and wherein a sparsity constraint of the tissue signal in the RPCA is in a wavelet-domain. form:

4. The method of claim 3, wherein the RPCA is an optimization problem of a
$${min}_{\{ L S \}} \{ {λ}_{1} {‖ L ‖}_{*} + {λ}_{2} {‖ S ‖}_{1} + \frac{1}{2} {‖ ( X - L - {W}^{H} S ) ‖}_{F}^{2} \}$$
where X, S, L represent received signals, tissue signals, and reverberation clutter artifact, respectively, ∥L∥_{*}, ∥S∥₁, and ∥ . . . ∥_{F}² are a nuclear norm, L1-norm and Frobenius norm, λ₁ and λ₂ are regularization parameters which affect the L and S, and W^{H} is an adjoint 2D wavelet transformation.

5. The method of claim 4, wherein an optimization method to solve the RPCA optimization problem includes at least one of Alternating Direction Method of Multiplier (ADMM), augmented Lagrange multiplier, fast alternating minimization, or iteratively reweighted least squares.

6. The method of claim 3, wherein the RPCA includes determining a singular value threshold, and wherein a singular value decomposition (SVD) is performed in which singular values below the determined singular value threshold are discarded.

7. The method of claim 1, wherein the first dimension rank of the tissue signals is different than the second dimension rank of the reverberation signals.

8. The method of claim 7, wherein the tissue signals have larger motion variability than the reverberation signals across the plurality of frames.

9. The method of claim 1, further comprising determining attenuation coefficient values to generate an attenuation coefficient map of the subject.

10. The method of claim 1, wherein the ultrasound imaging data includes at least one of in-phase/quadrature phase (IQ) data, post-beamformed radio frequency (RF) data, envelop data, or pre-beamformed channel data.

11. The method of claim 1, wherein the ultrasound imaging data are acquired in at least one of a fundamental or a harmonic imaging mode.

12. The method of claim 1, wherein the ultrasound imaging data are acquired during free breathing of the subject to impart motion to a tissue.

13. The method of claim 1, wherein the plurality of frames include at least one of 2D data or 3D data.

14. A system for reverberation signal suppression in ultrasound imaging of a subject, comprising:
a computer system configured to:
i) access ultrasound imaging data of a subject that includes a plurality of frames at different times and including tissue signals of a first dimension rank and reverberation signals of a second dimension rank;
ii) generate a region of interest (ROI) frames subset by determining a ROI for each frame in the plurality of frames;
iii) generate a spatiotemporal matrix from the ROI frames subset;
iv) separate the tissue signals from the reverberation signals in the spatiotemporal matrix using an adaptive method; and
v) generate an image of the subject with the reverberation signals suppressed by subtracting the separated reverberation signals from the ultrasound imaging data.

15. The system of claim 14, wherein the adaptive method includes at least one of a robust principal component analysis (RPCA), principal component analysis, singular value decomposition, non-parametric-based method, independent component analysis, or blind source method.

16. The system of claim 15, wherein the adaptive method is RPCA, and wherein a sparsity constraint of the tissue signal in the RPCA is in a wavelet-domain. form:

17. The system of claim 16, wherein the RPCA is an optimization problem of a
$${min}_{\{ L S \}} \{ {λ}_{1} {‖ L ‖}_{*} + {λ}_{2} {‖ S ‖}_{1} + \frac{1}{2} {‖ ( X - L - {W}^{H} S ) ‖}_{F}^{2} \}$$
where X, S, L represent received signals, tissue signals, and reverberation clutter artifact, respectively, ∥L∥_{*}, ∥S∥₁, and ∥ . . . ∥_{F}² are a nuclear norm, L1-norm and Frobenius norm, λ₁ and λ₂ are regularization parameters which affect the L and S, and W^{H} is an adjoint 2D wavelet transformation.

18. The system of claim 17, wherein an optimization method to solve the RPCA optimization problem includes at least one of Alternating Direction Method of Multiplier (ADMM), augmented Lagrange multiplier, fast alternating minimization, or iteratively reweighted least squares.

19. The system of claim 16, wherein the RPCA includes determining a singular value threshold, and wherein a singular value decomposition (SVD) is performed in which singular values below the determined singular value threshold are discarded.

20. The system of claim 14, wherein the first dimension rank of the tissue signals is different than the second dimension rank of the reverberation signals.

21. The system of claim 20, wherein the tissue signals have larger motion variability than the reverberation signals across the plurality of frames.

22. The system of claim 14, wherein the computer system is further configured to determine attenuation coefficient values to generate an attenuation coefficient map of the subject.

23. The system of claim 14, wherein the ultrasound imaging data includes at least one of in-phase/quadrature phase (IQ) data, post-beamformed radio frequency (RF) data, envelop data, or pre-beamformed channel data.

24. The system of claim 14, wherein the ultrasound imaging data are acquired in at least one of a fundamental or a harmonic imaging mode.

25. The system of claim 14, wherein the ultrasound imaging data are acquired during free breathing of the subject to impart motion to a tissue.

26. The system of claim 14, wherein the plurality of frames include at least one of 2D data or 3D data
